(19) United States
(12) Patent Application Publication
(10) Pub. No.: US 2011/0229598 A1
(43) Pub. Date: Sep. 22, 2011

(54) Enhanced lactoperoxidase system for treatment of milk products

(75) Inventor: Robert C. Musser, Woodbury, MN (US)

(73) Assignee: Land O'Lakes Purina Feed LLC, Shoreview, MN (US)

(21) Appl. No.: 13/046,162

(22) Filed: Mar. 11, 2011

Related U.S. Application Data
(60) Provisional application No. 61/315,224, filed on Mar. 18, 2010.
(60) Provisional application No. 61/352,971, filed on Jun. 9, 2010.

(51) Int. Cl. A23K 1/165; A23C 9/12; A23K 1/18; A61K 38/44; A61K 38/54

(52) U.S. Cl. 426/2; 426/61; 426/10; 424/94.4; 424/94.2

(57) ABSTRACT

The present invention relates to treatment of milk and milk products such as waste-milk with an enhanced lactoperoxidase system. The enhanced lactoperoxidase system is activated by the addition of a hydrogen peroxide source and an oxidizable agent, such as a halide to the milk to inactivate the bacterial pathogens. The enhanced lactoperoxidase system may be used alone or in conjunction with pasteurization to reduce or eliminate the bacterial load in milk products.

### CROSS-REFERENCE TO RELATED APPLICATION

The present application is based on and claims the benefit of U.S. provisional patent application Ser. No. 61/315,224, filed Mar. 18, 2010 and U.S. provisional patent application Ser. No. 61/352,971, filed Jun. 9, 2010, the content of which is hereby incorporated by reference in its entirety.

### FIELD

The present invention relates to the use of an enhanced lactoperoxidase system for treatment of milk products. In particular, the enhanced lactoperoxidase system is used for treatment of waste-milk that can then be used to feed calves.

### BACKGROUND

Farmers are faced with the challenge of raising healthy calves while trying to minimize the cost of feeding and caring for these animals. Dairy farmers generally have a supply of milk that is not saleable, commonly called waste-milk. Waste-milk can be non-saleable transition milk, mastitic milk or non-saleable antibiotic treated milk, i.e. milk from antibiotic treated animals, high somatic cell count milk that the producer has opted not to sell, or milk that is for any reason set aside to be fed to animals rather than sold for human consumption. This waste-milk had been used by dairy farmers to feed calves but concerns with the safety of this unpasteurized milk has led to the recommendation that unpasteurized waste-milk not be fed to calves. The risks of feeding unpasteurized milk to calves include transmission of infectious disease pathogens. Since the waste-milk is not saleable, the dairy farmer faces the unfortunate task of disposing of the waste-milk and/or using saleable raw milk or milk replacer to feed the calves. Alternatively, the farmer may choose to pasteurize the waste-milk, which adds new dimensions to proper management. Additionally, all of these choices lead to an increase in the costs incurred by a dairy farmer.

Currently many dairy farmers have some on site pasteurization equipment to pasteurize the waste-milk generated on the farm. These on-farm pasteurizers, however, are not as effective as the pasteurization of milk conducted in an off-site commercial dairy facility dedicated to pasteurization of milk for human consumption. This is because maintenance and cleaning of on-farm pasteurizers is not nearly as stringent as in commercial dairy facilities which process milk for human consumption. Additionally, the environment is not aseptic, containing bacteria loaded dust from dried manure and the like. Furthermore the bacterial load of waste milk is extremely high compared to saleable raw milk. Finally, due to the environment and handling/feeding practices, on-farm pasteurized milk is often re-inoculated with pathogens and spoilage organisms several hours prior to feeding.

In developing countries, the lack of appropriate refrigeration and storage facilities and inadequate transport systems compound the difficulties of preserving locally produced milk as well as delivering milk to processing facilities for pasteurization. A lactoperoxidase system has been employed in such cases to extend the shelf-life of milk. The lactoperoxidase anti-bacterial system is indigenous to milk due to the occurrence of lactoperoxidase (a naturally occurring enzyme in milk), low levels of hydrogen peroxide often introduced by common bacteria and thiocyanate, which naturally occurs at varying levels within milk. In developing countries the levels of hydrogen peroxide and thiocyanate are standardized by adding set amounts to further activate the indigenous lactoperoxidase in milk resulting in an effective method of milk preservation for delivery, without refrigeration, to dairy plants for pasteurization and further processing for human consumption. Thiocyanate, however, has not been approved by the Association of American Feed Control Officials, Inc. (AAFCO) for use in animals and thus cannot be used in waste-milk that may be fed to calves. Furthermore, the halide, iodide, has been shown to be much more effective than thiocyanate.

### SUMMARY

The present invention relates to milk products treated with an enhanced lactoperoxidase system. The lactoperoxidase system is activated by the addition of a hydrogen peroxide source and an oxidizable agent such as a halide to a milk composition to inactivate spoilage organisms and bacterial pathogens. The hydrogen peroxide can be generated by the addition of glucose oxidase and glucose to the milk composition. The enhanced lactoperoxidase system may be used in conjunction with pasteurization to greatly reduce or eliminate the bacterial load in waste-milk. The present invention also relates to methods of treating waste-milk to sufficiently kill the bacteria using the enhanced lactoperoxidase system such that the waste-milk is acceptable as feed for calves.

In one aspect, the present invention includes a milk product comprising a milk composition and LP system components, wherein the LP system components comprise lactoperoxidase, glucose oxidase, glucose and an oxidizable agent.

In another aspect, the present invention includes an LP system activation add pack for milk compositions, the add pack components comprise glucose oxidase, glucose and an oxidizable agent wherein addition of the components of the add pack inactivates the bacterial pathogens in the milk composition.

In yet another aspect, the present invention includes a method of treating a milk composition comprising activating an enhanced lactoperoxidase system by adding lactoperoxidase system components comprising glucose oxidase, glucose and an oxidizable agent.

In a further aspect, the present invention includes a method of feeding calves comprising providing a milk composition treated with an enhanced lactoperoxidase system. The treatment comprises activation of an enhanced lactoperoxidase system by addition of enhanced lactoperoxidase system components comprising glucose oxidase, glucose and an oxidizable agent.

In another further aspect, the present invention includes a method of reducing the spread of Johne's disease in animals. The method includes feeding the animals milk products treated with an enhanced lactoperoxidase system wherein the treatment comprises addition of components needed to activate the lactoperoxidase system, the components comprising glucose, glucose oxidase and a halide.

### BRIEF DESCRIPTION OF THE DRAWINGS

FIG. 1 is a graph of the standard plate count of waste milk, pre pasteurization, post pasteurization and at the time of last calf feeding.

FIG. 2 is images of plates showing plaques under different treatment conditions.

### DETAILED DESCRIPTION

The present invention relates to an enhanced antibacterial system for milk and milk related products. The preservation system is an enhanced lactoperoxidase system and involves lactoperoxidase, hydrogen peroxide and a halide, preferably iodide. Lactoperoxidase is an enzyme naturally occurring in the whey protein of milk and can oxidize molecules such as a halide in the presence of hydrogen peroxide. This preservation system advantageously is a natural and highly effective system, and may be used alone or in conjunction with pasteurization.

The present invention includes the addition of a source(s) that provide hydrogen peroxide and an oxidizable agent such as a halide to a milk composition in order to activate the lactoperoxidase antibacterial system. The hydrogen peroxide source can include the addition of glucose oxidase and glucose because glucose oxidase oxidizes the glucose to form hydrogen peroxide. Lactoperoxidase, in the presence low levels of hydrogen peroxide, oxidizes the halide generating a potent bactericidal system that can aid in milk preservation and destruction of pathogens. The enhanced lactoperoxidase system may also be effective against a number of bacteria that are particularly difficult to inactivate, such as Mycobacterium avium sub. paratuberculosis (MAP) which causes Johne's disease—a chronic wasting disease in cattle, and for which pasteurized waste-milk can be a vehicle for infection, since the organism has been shown to survive typical pasteurization. The lactoperoxidase system may also be employed to greatly reduce or prevent re-inoculation and growth of pathogens in pasteurized waste-milk. Such pathogens might include E. coli, Salmonella, Clostridium perfringens, and the like, which have often proven deadly to calves.

The enhanced lactoperoxidase system (LP) described herein can be beneficial for preserving milk products, for example, waste-milk and/or colostrum and reducing the occasion of milk or milk products being a vehicle for pathogens. Milk products referred to herein can include milk and milk-related products. Milk products can include, for example, raw milk, pasteurized milk, waste-milk, colostrum, milk balancer products and the like. Waste-milk referred to herein relates to any milk that is generally discarded and deemed not suitable for human consumption due to the milk being obtained from mastitic animals, antibiotic treated animals, transition cows and the like, or milk that is for any reason set aside to be fed to animals rather than sold for human consumption. The LP system is particularly preferred for use in treatment of waste-milk. Although the present invention is described below with respect to waste-milk embodiments, other milk products can also be treated in a similar manner and are all within the scope of this invention. Milk or milk-related products treated with the LP system can be suitable for consumption by animals and/or humans.

In the present invention, the activation of the LP system can include addition of glucose, glucose oxidase and an oxidizable agent such as a halide. Halides can include, for example, chloride, fluoride, bromide and iodide. In preferred embodiments, iodide is used as the halide component. The glucose and the glucose oxidase are generally used to generate the hydrogen peroxide, but other peroxide sources may be employed, such as percarbonate, magnesium peroxide or a drip H₂O₂ application, the use of which is also within the scope of this invention.

Generally, sufficient endogenous lactoperoxidase is present in the milk products for inactivation of the pathogens. Other peroxidases exist which may be added to waste-milk fed to animals, which will enhance the antibacterial system. Other peroxidases can include, for example, horseradish peroxidase, fungal peroxidases and the like. These peroxidases may be isolated and concentrated from their natural source, or manufactured using recombinant DNA technology. The use of these and the like, as part of an antibacterial system in waste-milk is within the scope of this invention. Additional exogenous lactoperoxidase may also be added to enhance the antibacterial system.

The use of glucose and glucose oxidase is advantageous because they are both approved products for use in animals and are easily obtainable and non-toxic, and release of the peroxide is slow, occurring over an extended period of time. The glucose, glucose oxidase and halide may be in liquid form or in powdered form. A mixture of liquid components and powdered components are also within the scope of this invention.

The amount of glucose, glucose oxidase and halide that are used to treat milk compositions can vary. Generally, the amount of glucose used in the waste-milk is between about 0.5 grams per liter and about 10.0 grams per liter. Preferably, the amount of glucose used in the waste-milk is between about 0.75 grams per liter and about 7.5 grams per liter and more preferably, between about 1.0 and about 1.1 grams per liter. The amount of glucose oxidase used in the waste-milk can vary and preferably is between about 0.01 grams per liter and about 0.1 grams per liter of a 10,000 GOD Units/gram glucose oxidase product. More preferably, the amount of glucose oxidase used in the waste-milk is between about 0.03 grams per liter and about 0.06 grams per liter of a 10,000 GOD Units/gram glucose oxidase product. The concentration of the halide used in the waste-milk can vary. For example, in embodiments using iodide, the concentration is generally between about 0.1 ppm and about 10 ppm. Preferably, the concentration of the iodide in the waste-milk is about 4 ppm. Concentrations outside of these ranges are also within the scope of the invention.

In some embodiments, salts of organic acids such as sorbates, benzoates and propionates may also be added to the waste-milk. The organic acids can act synergistically with the components of the LP system to enhance the inactivation of the bacterial spoilage organisms and pathogens in the waste-milk. The amount of organic acid used can be between about 0.05 percent by weight and about 0.2 percent by weight. Preferably, the amount of organic acid used is about 0.075 percent. Amounts of organic acid outside of this range are also within the scope of this invention.

Treatment of milk products with the LP system can aid in preservation of the milk product by inactivating spoilage organisms and bacterial pathogens in the milk. Pasteurization of the milk, especially on-farm pasteurization of waste-milk, can reduce the number of pathogens to some extent but a significant number of pathogens may still be present or re-inoculation may occur due to unsanitary conditions, so that growth occurs in the excellent medium of poorly pasteurized waste milk. Pathogen levels of E. coli and Salmonella can grow at alarming rates, doubling their population every 20 minutes, so that last calves fed may be at risk of infection. Example 1, below, shows these results from pasteurization and demonstrates the need for a better method of reducing the bacterial pathogens and spoilage organisms.

A variety of organisms can be inactivated using the LP system in milk products including, for example, E. coli, Salmonella, Clostridium perfringens, Mycoplasma bovis, MAP and the like. Generally, treatment of waste-milk with the LP system can inactivate at least about 50 percent of the spoilage organisms and bacterial pathogens found in the milk. In some embodiments, treatment of waste-milk with the LP system can cause a reduction of at least about 2-fold, and in some preferred embodiments, the LP system can cause a reduction of at least about 10-fold of the spoilage organisms and bacterial pathogens found in the milk. Reduction of spoilage organisms greater than about 10-fold are also within the scope of the invention. When used in conjunction with on-farm pasteurization these numbers can greatly increase to a multiple log reduction of spoilage organisms and destruction of all pathogens. The combination of on-farm pasteurization with the LP system treatment can reduce the spoilage organisms by more than about 4 log (10,000 fold) reduction, preferably more than about 5 log (100,000 fold) reduction. In some preferred embodiments, the combination of on-farm pasteurization with the LP system effectively destroyed all of the pathogens.

The LP system can also increase the shelf-life of the waste-milk when used in conjunction with on-site pasteurization. The shelf-life of the waste-milk that has been pasteurized on-site and also treated with the LP system can be in excess of about 12 hours at 40° C. and in excess of about 7 days at 4.5° C., whereas on-farm pasteurized waste milk has been known to sour within 3 hours at 40° C. The LP system can increase the shelf-life of the waste-milk even without pasteurization. The shelf-life of LP system treated waste-milk can be greater than about four hours. In some preferred embodiments the shelf-life of LP system treated waste-milk can be greater than about eight hours.

The present invention also includes methods of treating milk compositions with the LP system. Milk compositions may be treated with the LP system prior to pasteurization, during pasteurization or after pasteurization. In activating the enhanced LP system, the components of the LP system may be added together or they may be added sequentially. In one embodiment, the iodide and glucose are added at a desired concentration first from within a balancer. The glucose oxidase is then added as an add-pack to complete activation of the lactoperoxidase. Generally, as the glucose oxidase dissolves and moves about within the milk, it oxidizes the glucose to generate hydrogen peroxide and gluconic acid. The lactoperoxidase then acts on the hydrogen peroxide and then the iodide. The oxidized iodide products of this reaction act as potent bactericidal agents in the milk products.

The milk compositions may be treated with the LP system at various temperatures. Generally, the milk compositions are treated with the LP system at temperatures where the glucose oxidase is the most active. Preferably, waste-milk is treated with the LP system at temperature between about 4° C. and about 50° C. More preferably, the waste-milk is treated with the LP system between about 38° C. and about 45° C. At the lower temperatures, the glucose oxidase can be functional, but not fully active. As the temperature increases, the activity of the glucose oxidase can increase. Adding the glucose oxidase at temperatures above 50° C. can denature the enzyme. The milk compositions can be treated with the LP system at higher temperatures using other peroxide sources such as percarbonate. About seventy percent of indigenous lactoperoxidase can survive pasteurization.

In some embodiments, the LP system is added prior to pasteurization and the temperature is gradually increased to allow for increased enzymatic activity prior to destruction of the glucose oxidase during the pasteurization process. Activity of the LP system can persist for some time beyond the destruction of the glucose oxidase. The LP system activity may be present in the waste-milk until it is consumed by an animal. Antibacterial activity may continue within the abomasum prior to digestion of the milk. Generally, the waste-milk can be treated with the LP system for at least about 20-600 minutes, preferably at least about 30 to 120 minutes. The treatment with the LP system can be prior to refrigeration, storage or feeding of the milk. It may also be prior to pasteurization, or after pasteurization of the milk but when it has cooled to the appropriate temperature for optimal enzymatic activity. All storage or handling should be accompanied by mild agitation as in that which is used to prevent creaming out of the milk fat, because the efficiency of enzymes is related to random collisions related to motion.

The present invention also includes treating milk compositions to inactivate the Mycobacterium avium subsp. Paratuberculosis (MAP) bacteria that may be present in the milk compositions. The MAP bacteria in milk are difficult to eliminate and seem to survive pasteurization in some cases. MAP is known to be causative agent of Johne's disease in cattle and other ruminants. Thus, inactivation or elimination of MAP in milk can reduce the incidence of the disease, especially considering susceptibility is high during the early stages of the animal's life. Cow's milk is one of the primary vehicles for dissemination of MAP, both through MAP being passed through the mammary gland and due to on-farm fecal contamination of the milk. MAP in milk can be eliminated by treating the milk with the LP system in combination with pasteurization as described above. This is demonstrated in Tables 10 and 11 below. The use of the enhanced LP system with a halide, preferably, iodide can be particularly effective against MAP, since the LP system mimics the highly effective peroxidase, peroxide, halide (PPH) system which is used by phagocytes to destroy bacterial invaders. Phagocytes which MAP organisms are able to invade are those which have lost this PPH system. The enhanced LP system is advantageously more effective against MAP and other pathogens, for example, than the lactoperoxidase system using thiocyanate or a chloride. The present invention may also include treating waste-milk with the LP system to inactivate Cryptosporidium parvum, a protozoan parasite that can cause life-threatening diarrhea in calves.

The present invention includes any milk products or milk-related products treated with the LP system, including, for example, treated waste-milk, raw and pasteurized milk, milk balancer and/or colostrum. The treated milk products include the LP system and reduced numbers of bacterial pathogens. The milk products may or may not have been pasteurized prior to, during or after treatment with the LP system. In preferred embodiments, the treated milk products have reduced numbers of the MAP, E. coli, Salmonella, Clostridium perfringens, Mycoplasma bovis and similar bacteria or organisms. The milk products even after storage have reduced numbers of bacterial and/or parasitic pathogens.

In one exemplary embodiment, the present invention includes treatment of a waste-milk composition with the LP system. The waste-milk product after treatment with the LP system can be appropriate for feeding calves. The waste-milk product may or may not be pasteurized. Preferably, the LP system uses iodide as an added component of the LP system. The pathogen load of the waste-milk treated with the LP system is significantly lower than the pathogen load of the untreated waste-milk, whether the LP system is used in conjunction with pasteurization or without pasteurization.

In another exemplary embodiment, the present invention also includes a milk balancer product. The milk balancer can include the components of the LP system and may also contain additional nutritional supplements. A milk balancer is a powdered supplement, similar to milk replacer, but it is designed to be added to waste milk to balance the nutrition of the waste-milk to optimal levels for dairy calves. Dairy calves are reared artificially, unable to nurse as desired, and live in a more controlled environment so their optimal nutritional requirements are different than what is provided by milk alone. A balancer generally includes protein, fat, and other nutrients such as vitamins and minerals, as well as neutraceuticals, approved medications and other functional ingredients. The amounts of nutritional supplements present in a milk balancer product can vary and depend on the specific use of the milk balancer, but its intent would be to optimize the nutrition being delivered to the calf. In one exemplary embodiment, the milk balancer product may include the glucose, glucose oxidase, iodide, protein and fat. The amount of protein in the milk balancer can be between about 15 percent by weight and about 30 percent by weight, preferably between about 24 percent by weight and about 28 percent by weight. The amount of fat in the milk balancer can be between about 2 percent by weight and about 15 percent by weight, preferably between about 8 percent by weight and about 12 percent by weight.

The present invention can also include kits or add packs with components that can activate the LP system. The kits or add packs can include activation components such as glucose, glucose oxidase and a halide. In some embodiments, additional peroxidase such as additional lactoperoxidase or other peroxidases as described herein can also be included. Other components such as other hydrogen peroxide sources, salts of organic acids and the like may also be included. The components may be packaged individually or they may be combined to form a mixture or mixtures that can be added to the milk compositions. The components can be added to the milk compositions prior to storage and/or prior to consumption by animals or humans.

The present invention also includes a method of feeding calves that enhances the growth characteristics of the calves. Other animals that may also be fed the LP system treated milk can include, for example, lambs, kids, foals, and other young animals that can be fed milk. The calves can be fed waste-milk or pasteurized waste-milk treated with the LP system. Alternatively, the calves may be fed waste-milk or pasteurized waste-milk that has been combined with a milk balancer product. The milk balancer product includes the LP system and nutritional supplements as described above or waste milk treated with the LP system prior to pasteurization, to which a balancer is added. The calves fed the waste-milk treated with the LP system described herein can have improved growth and or health profiles.

In another embodiment, the present invention can also include a method of aiding in the prevention of Johne's disease. The method can reduce the spread and/or occurrence of Johne's disease by inactivating MAP that may be present in milk products. This in turn can reduce the occurrence and exposure of MAP to animals. The method includes feeding animals milk products treated with the LP system described herein.

### EXAMPLES

### Example 1

The effect of pasteurization on waste-milk in a large number of dairies was evaluated. This study demonstrates the need for a better method or augmentation of the method of reducing or eliminating bacteria. Samples from over 200 dairies were collected. 217 samples were analyzed. The standard plate counts (SPCs) were obtained pre-pasteurization, post-pasteurization and later as the last calf was fed. The time elapsed until the last calf fed was between 1 to 4 hours.

TABLE 1

| | Pre-past. | Post-past. | Last calf-fed |
|---|---|---|---|
| 10K to 50K | 71 | 147 | 125 |
| 50K to 500K | 97 | 45 | 57 |
| 500K to 1000K | 24 | 12 | 12 |
| 1000K to 2000K | 51 | 36 | 44 |

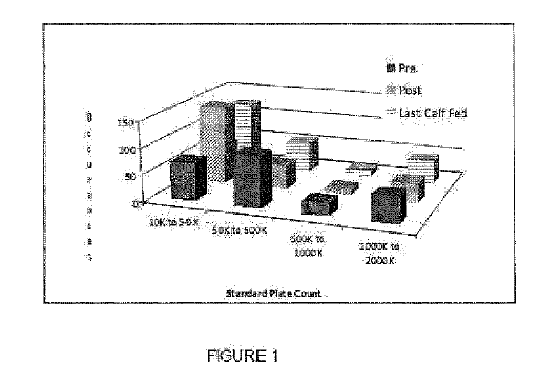

As can be seen in Table 1 and shown in FIG. 1, there are significant number of bacteria as indicated by SPCs in the samples examined prior to pasteurization. When the waste-milk is pasteurized (post-past), the SPCs in a number of these samples were reduced to between 10K and 50K. As time elapses and samples were taken as the last calf was fed, the number of samples with SPCs in the higher levels starts to increase indicating that the effect of pasteurization is already starting to be compromised. This study indicates the need for a more effective method of eliminating bacteria.

### Example 2

The effect of the enhanced lactoperoxidase system (LP system) in pasteurized milk, store bought milk and saleable raw milk was evaluated using the standard plate counts. Plate counts were obtained initially and after 4.5 hours at 38° C. The plate counts were done using the methods described, for example, in FDA:BAM (online), Aerobic Plate Count, January 2001.

The amounts of the LP system components used are as shown in Table 2.

TABLE 2

| Components | LP System, g/L |
|---|---|
| KI (potassium Iodide) | 0.0102 |
| GLOX* | 0.075 |
| glucose | 7.5 |
| K-sorbate | 1.5 |

*GLOX = Glucose oxidase from Novozyme, brand name Gluzyme 10,000

The results of the standard plate counts (SPC) are shown in Table 3.

TABLE 3

| | | Initial SPC | SPC @ 4.5 hrs 38° C. |
|---|---|---|---|
| | | (cfu/ml) | (cfu/ml) |
| Pasteurized | Control | 29,000 | 36,000,000 |
| waste milk | LP system | 29,000 | 1,100 |
| Store | Control | 29 | 610 |
| bought milk | LP system | 29 | <1 |
| Saleable Raw | Control | 34,000 | 3,800,000 |
| milk | LP system | 34,000 | 610 |

The use of the LP system greatly enhances the reduction of the colony forming units (cfu) in the samples after 4.5 hours at 38° C.

### Example 3

The effect of the LP system in pasteurized milk, store bought milk and saleable raw milk was evaluated using the standard plate counts but with lower levels of glucose and potassium sorbate compared to the levels used in Example 2 above. Plate counts were obtained initially and after 4.5 hours at 38° C. as indicated above.

The amounts of the LP system components used are as shown in Table 4.

TABLE 4

| Components | LP System, g/L |
|---|---|
| KI (potassium Iodide) | 0.0102 |
| GLOX* | 0.075 |
| glucose | 1.75 |
| K-sorbate | 1.25 |

*GLOX = Glucose oxidase from Novozyme, brand name Gluzyme 10,000

The results of the standard plate counts (SPC) used are shown in Table 5.

TABLE 5

| | | Initial SPC | SPC @ 4.5 hrs 38° C. |
|---|---|---|---|
| | | (cfu/ml) | (cfu/ml) |
| Saleable | Control | 7100 | 1,800,000 |
| raw milk | LP system | 7100 | 4000 |
| | (as in Example | | |
| | 2) | | |
| | LP system as in | 7100 | 2500 |
| | Table 4 | | |
| Pasteurized | Control | 43,000 | 34,000,000 |
| Waste milk | LP system | 43,000 | 10,000 |
| | (as in Example | | |
| | 2) | | |
| | LP system as in | 43,000 | 4700 |
| | Table 4 | | |

The use of the LP system greatly enhances the reduction of the colony forming units (cfu) in the samples after 4.5 hours at 38° C.

### Example 4

The effect of the LP System on the growth of E. coli, Salmonella and Clostridium perfringens in milk was evaluated. The concentrations of the LP system components used are shown below in Table 6.

TABLE 6
LP System Activator Concentrations, Grams per Liter Raw Milk

| | LP System, g/L | LP System 60% g/L |
|---|---|---|
| KI (potassium Iodide) | 0.0102 | 0.0061 |
| GLOX* | 0.075 | 0.045 |
| glucose | 1.75 | 1.05 |
| K-sorbate | 1.25 | 0.75 |

*GLOX = Glucose oxidase from Novozyme, brand name Gluzyme 10,000

### Culture Preparation

Culture preparations consisted of 5 E. coli strains, 5 Salmonella strains and 5 Clostridium perfringens strains. All of the strains were received from Wisconsin Veterinary Diagnostic Laboratory and were confirmed calf pathogens. E. coli and Salmonella strains were grown in nutrient broth at 35° C. and Clostridium perfringens was grown in Reinforced Clostridium broth at 37° C. anaerobically. The five strains of each organism were combined and used in separate mixed preparations for growth evaluations.

The milk samples were evaluated without the LP system, with the LP system and with the LP system at a level of 60%. The samples were evaluated initially (0 hours) and at 3 hours.

### Evaluation of Growth

Milk Samples were dispensed into sterile test tubes and inoculated with a mixed culture that consisted of E. coli, Salmonella, or Clostridium perfringens. The target inoculation level was 10⁴ CFU/mL. Cultures were diluted in sterile saline before use in inoculation to minimize growth media carry over to test variables. Ratio of inoculum volume to total test suspension volume was less than 0.1%. The culture tubes were incubated for 3 hours in a 39° C. agitating water bath at 60 oscillations per minute. The growth of bacteria was enumerated by plating method at time 0 and 3 hours.

All milk with LP System variables had an impact on the growth of total bacteria (Standard Plate Count) as shown in Table 7. The bacteria counts increased 2.5 log in the milk without LP System after 3 hr incubation at 39° C. However, no significant change in bacteria counts was observed in milk with LP System treatment.

TABLE 7

Total bacteria counts (cfu/mL)

| Variable | 0 hr | 3 hr |
|---|---|---|
| Milk without LP | 16,000 | 4,300,000 |
| SYSTEM | | |
| Milk with LP SYSTEM | 16,000 | 14,000 |
| Milk with LP SYSTEM | 16,000 | 65,000 |
| 60% | | |

Table 8 shows the results of the LP system effect on the different bacteria. All milk with LP System variables decreased the counts of E. coli and Salmonella after 3 hr incubation at 39° C. as shown in Table 8. However, in the milk without LP System, the counts of E. coli and Salmonella increased 1.8 log.

For Clostridium perfringens, no significant change in the counts was observed in milk without LP System and milk with LP System after a 3 hour incubation. See Table 8. However, samples for initial inoculation counts of Clostridium perfringens in the LP System treatments were taken 30 minutes after LP activation, rather than prior to inoculation; this could have affected the outcome.

TABLE 8

| | | | | | Clostridium | |
|---|---|---|---|---|---|---|
| | | | Salmonella | | perfringens | |
| | E. coli (cfu/mL) | | (cfu/mL) | | (cfu/mL) | |
| Variable | 0 hr | 3 hr | 0 hr | 3 hr | 0 hr | 3 hr |
| Milk without | 10,000 | 560,000 | 6,000 | 390,000 | 60 | 70 |
| LP SYSTEM | | | | | | |
| Milk with LP | 9,800 | 5,100 | 4,900 | 1,700 | 10 | 10 |
| SYSTEM | | | | | | |
| Milk with LP | 10,000 | 2,700 | 6,000 | 1,400 | 10 | 10 |
| SYSTEM 60 | | | | | | |

### Example 5

The effect of the LP System on viability of Mycobacterium avium subsp. Paratuberculosis (MAP) cells in raw milk under less than optimal pasteurization temperatures (simulating what may occur during on-farm pasteurization of waste milk) was evaluated. Typically pasteurization time and temperature for batch pasteurization is performed at 62.5° C. or 145° F. for 30 min. In this study, temperatures of 39, 53 and 56.5° C. for 30 minutes were used to simulate less than optimal pasteurization conditions. These conditions were evaluated with and without the LP system.

FASTPlaque-MAP Assay

### 1. Preparation of the MAP Strains

Three strains of MAP were received from National Animal Disease Center (NADC), USDA, Ames, Iowa. Two strains, cow 167 and cow 509 P+1, were isolated from a clinical cow and the other strain, k-10 P4 was a reference strain. The strains were grown to an OD₅₄₀ of 0.26 after a 5 wk incubation at 37° C. The concentration of MAP culture was adjusted to 10⁵ to 10⁶ CFU/ml for product inoculation. The three strains were combined and used as a mixture in this study.

2. Immunomagnetic Separation (IMS) from Milk

50 ml of milk were centrifuged at 2500×g for 15 min at 5° C. The whey fraction and cream were discarded and the pellet was resuspended in 3 ml NOA-supplemented Media Plus. The sample was recentrifuged for 10 min at 2500×g at 5° C. and pellet resuspended in 1 ml NOA-supplemented Media Plus. IMS was applied to the whole sample. 5 μl of each of the two types of coated beads (aMp3 and aMptD) were dispensed into empty 1.5 ml Eppendorf tubes. Prepared milk samples were transferred to tubes containing the magnetic beads and each tube was vortexed briefly.

Immunocapture step: the contents of the tubes were mixed gently for 30 min at room temperature on a Stuart rotator mixer at 8 rpm. Magnetic separation step: tubes were transferred to magnetic rack and beads were separated for 10 min. The rack was rocked back and forth half-way through the separation period. The sample was carefully pipetted leaving the beads adhering to the back of the tube. Wash steps: beads were washed twice with 1 ml wash buffer (PBS-T20), and separated on the magnet for 2 min between washes. The beads were resuspended in 1 ml PBS-T20 and 300 μl was taken for enumeration on HEYM slopes. Magnetic separation was performed again and remaining beads resuspended in 700 μl NOA-supplemented Media Plus and incubated overnight at 4° C. for phage assay.

### 3. FASTPlaque-MAP Assay (PFU/ml): Per Assay Kit

Samples were warmed to room temperature or placed at 37° C. for 15 min. 100 μl Actiphage was added and the sample incubated for 2 h at 37° C. 100 μl Virusol was added and the sample incubated for 5 min on the bench. The sample was mixed thoroughly after addition of Virusol to ensure entire internal surface of tube is wetted. 5 ml FP Media Plus was added to stop virucide (total volume of sample was 6.2 ml) and tubes were inverted once and samples placed back in incubator for a further 1 h. 10-fold dilutions of the sample were prepared by removing 0.5 ml from sample and mixing with 4.5 ml FP-MAP Media Plus. 0.5 ml was discarded from last dilution (volume of dilution tube is 4.5 ml). 1 ml sensor cells was added to the remainder of the original mixture (volume=5.7 ml) and dilution tube (4.5 ml). Each of the samples was transferred to plates and mixed with 5 ml FP-MAP agar. Plates were inverted and incubated overnight at 37° C. Plaques were counted after overnight incubation.

The titer for MAP culture was about 7.5×10⁵ CFU/ml. Immunomagnetic separation (IMS) was used as a decontamination step to reduce milk components and background microflora. The protocol and beads were purchased from Lab21 Limited, Cambridge, United Kingdom. FASTPlaque-MAP assay was used to detect viable counts of MAP in milk samples. Table 9 shows the amount of the LP system components used.

TABLE 9
LP System Activator Concentrations, Grams per Liter Raw Milk

| | LP System, g/L |
|---|---|
| KI (potassium Iodide) | 0.0102 |
| Glox | 0.075 |
| glucose | 1.75 |
| K-sorbate | 1.25 |

Detection of live MAP was accomplished by using the FASTPlaque-MAP assay and the MAP culture enumeration method (MPN method)

### Treatments Design

Concentrated MAP cultures (appropriately 10⁵ to 10⁶ CFU/ml) were inoculated into raw milk to reach target spiking concentrations of 10³ to 10⁴ CFU/ml. Raw milk alone was used as an uninoculated control. For each variable, duplicate samples with 3 dilutions (10⁴ CFU/ml, 10³ CFU/ml and 10² CFU/ml) were tested. The treatment in each reaction tube is shown in Table 10 below.

TABLE 10

| Reaction vessel # | Treatment |
|---|---|
| 1 | Assay positive control |
| 2 | Assay negative control |
| 3-10 | MAP culture 10⁶ CFU/ml, 10⁴ CFU/ml, |
| | 10³ CFU/ml, 10² CFU/ml |
| 11 | Raw milk control |
| 12-17 (Variable 1, Positive | Inoculated raw milk with 10⁴ CFU/ml MAP |
| Control)) | |
| 18-23 (Variable 2) | Inoculated raw milk with 10⁴ CFU/ml MAP |
| | LP SYSTEM treatment |
| | 38° C. for 3.5 hr |
| 24-29 (Variable 3) | Inoculated raw milk with 10⁴ CFU/ml MAP |
| | NO LP SYSTEM treatment |
| | 51° C. for 30 min |
| 30-35 (Variable 4) | Inoculated raw milk with 10⁴ CFU/ml MAP |
| | LP SYSTEM treatment |
| | 51° C. for 30 min |
| 36-41(Variable 5) | Inoculated raw milk with 10⁴ CFU/ml MAP |
| | No LP SYSTEM treatment |
| | 56.5° C. for 30 min |
| 42-47(Variable 6) | Inoculated raw milk with 10⁴ CFU/ml MAP |
| | LP SYSTEM treatment |
| | 56.5° C. for 30 min |

### MAP Culture Enumeration (MPN Method)

300 μl IMS beads in PBS-T20 solution (see Part 1, IMS procedure step 9) was diluted and struck onto Herrold's egg yolk medium (HEYM) containing 2 μg/ml mycobactin J. Colony count (CFU/ml) was determined after a 6-week incubation at 37° C.

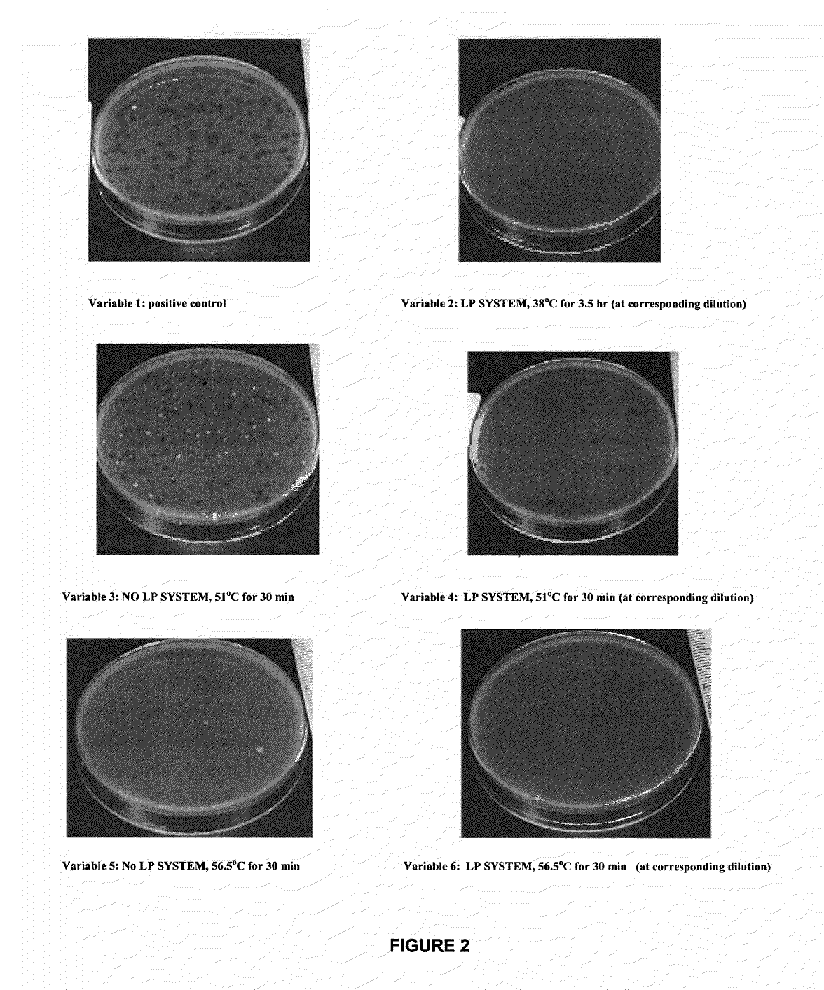

Results are shown in Table 11 (FASTPlaque-MAP Assay Results) and Table 12 (MPN Method Results) below. The representative phage pictures of 6 variables are shown in FIG. 2.

The most probable number (MPN) method was used to estimate the number of MAP cells per ml of sample. The MPN estimation was determined using 3 series dilutions with 3 slants per dilution level (9 tubes).

TABLE 11

| | | Plaque | | | | Estimated | |
|---|---|---|---|---|---|---|---|
| | | counts/ | | Log₁₀PFU/ | | CFU counts/ | |
| | | 50 ml | | 50 ml | Log | 50 ml* | |
| Variables | Description | Rep 1 | Rep 2 | (average) | reduction | Rep 1 | Rep 2 |
| 1 | Inoculated raw milk | 1900 | 1200 | 3.19 | | 2100 | 1300 |
| (Positive | with 10⁴ CFU · ml | | | | | | |
| Control) | MAP | | | | | | |
| 2 | Inoculated raw milk | 21 | 30 | 1.41 | −1.78 | 23 | 33 |
| | with 10⁴ CFU · ml | | | | | | |
| | MAP | | | | | | |
| | LP SYSTEM | | | | | | |
| | treatment | | | | | | |
| | 38° C. for 3.5 hr | | | | | | |
| 3 | Inoculated raw milk | 93 | 780 | 2.64 | −0.55 | 100 | 850 |
| | with 10⁴ CFU/ml | | | | | | |
| | MAP | | | | | | |
| | NO LP SYSTEM | | | | | | |
| | treatment | | | | | | |
| | 51° C. for 30 min | | | | | | |
| 4 | Inoculated raw milk | 84 | 68 | 1.88 | −1.31 | 92 | 74 |
| | with 10⁴ CFU/ml | | | | | | |
| | MAP | | | | | | |
| | LP SYSTEM | | | | | | |
| | treatment | | | | | | |
| | 51° C. for 30 min | | | | | | |
| 5 | Inoculated raw milk | 24 | 20 | 1.34 | −1.85 | 26 | 20 |
| | with 10⁴ CFU/ml | | | | | | |
| | MAP | | | | | | |
| | No LP SYSTEM | | | | | | |
| | treatment | | | | | | |
| | 56.5° for 30 min | | | | | | |
| 6 | Inoculated raw milk | 1 | 1 | 0 | −3.19 | 1 | 1 |
| | with 10⁴ CFU/ml | | | | | | |
| | MAP | | | | | | |
| | LP SYSTEM | | | | | | |
| | treatment | | | | | | |
| | 56.5° C. for 30 min | | | | | | |

*Based on FASTPlaque-MAP assay, CFU counts can be estimated using plaques numbers. CFU/ml = No. Plaques * 1.09.
Notes:
Article “Rapid assessment of the viability of Mycobacterium avium subsp. paratuberculosis cells after heat treatment, using an optimized phage amplification assay.” Author Irene Grant indicated plaque counts obtained using FASTPlaque-MAP assay were not significantly different from colony counts on HEYM plates.

TABLE 12

| | | MPN estimate/50 ml | | | |
|---|---|---|---|---|---|
| | | | 95% confidence | | 95% confidence |
| Variables | Description | Rep 1 | limit | Rep 2 | limit |
| 1 | Inoculated raw milk with | >37000 | (14000, —) | >37000 | (14000, —) |
| (Positive | 10⁴ CFU/ml MAP | | | | |
| Control) | | | | | |
| 2 | Inoculated raw milk with | 15000 | (3000, 67000) | 15000 | (3000, 67000) |
| | 10⁴ CFU/ml MAP | | | | |
| | LP SYSTEM treatment | | | | |
| | 38° C. for 3.5 hr | | | | |
| 3 | Inoculated raw milk with | 37000 | (6000, 140000) | 37000 | (6000, 140000) |
| | 10⁴ CFU/ml MAP | | | | |
| | NO LP SYSTEM | | | | |
| | treatment | | | | |
| | 51° C. for 30 min | | | | |
| 4 | Inoculated raw milk with | 15000 | (3000, 67000) | 15000 | (3000, 67000) |
| | 10⁴ CFU/ml MAP | | | | |
| | LP SYSTEM treatment | | | | |
| | 51° C. for 30 min | | | | |
| 5 | Inoculated raw milk with | 1200 | (290, 3100) | 970 | (290, 3100) |
| | 10⁴ CFU/ml MAP | | | | |
| | No LP SYSTEM | | | | |
| | treatment | | | | |
| | 56.5° C. for 30 min | | | | |
| 6 | Inoculated raw milk with | No Growth | (—, 320) | No Growth | (—, 320) |
| | 10⁴ CFU/ml MAP | | | | |
| | LP SYSTEM treatment | | | | |
| | 56.5° C. for 30 min | | | | |

Both FASTPlaque-MAP assay and MPN method indicated that the most effective treatment against MAP cells in raw milk was the LP System at 56.5° C. for 30 min. FASTPlaque-MAP assay showed that a treatment with the LP System at 56.5° C. for 30 min resulted in a 3.34 log₁₀ CFU/50 ml reduction of MAP over the untreated raw milk control sample.

### Example 6

The effect of lactoperoxidase system on viability of MAP cells in raw milk under less than optimal pasteurization temperatures (simulating what may occur during on-farm pasteurization of waste milk) was evaluated. Typically, pasteurization time and temperature for batch pasteurization is 62.5° C. or 145° F. for 30 min. This trial looked at temperatures of 53, 56.5 and 58.9° C. for 30 minutes with and without the LP system 60%. Detection of live MAP was accomplished using the FASTPlaque-MAP assay. The protocols for FASTPlaque-MAP assay are as described above in Example 5. The amounts of LP system components added are shown in Table 13.

TABLE 13
LP System Activator Concentrations, Grams
per Liter Raw Milk

| | LP System 60% g/L |
|---|---|
| KI (potassium | 0.0061 |
| Iodide) | |
| GLOX* | 0.045 |
| glucose | 1.05 |
| K-sorbate | 0.75 |

*GLOX = Glucose oxidase from Novozyme, brand name Gluzyme 10,000

Concentrated MAP cultures (appropriately 10⁵ to 10⁶ CFU/ml) were inoculated into raw milk to reach target spiking concentrations of 10³ to 10⁴ CFU/ml. Raw milk was used as an uninoculated control. For each variable, duplicate samples with 3 dilutions (10⁴ CFU/ml, 10³ CFU/ml and 10² CFU/ml) were tested. The treatment in each of the tubes are as shown in Table 14.

TABLE 14

| Reaction vessel # | Treatment |
|---|---|
| 1 | Assay positive control |
| 2 | Assay negative control |
| 3-12 | MAP culture 10⁶ CFU/ml, 10⁵ CFU/ml, |
| | 10⁴ CFU/ml, 10³ CFU/ml, 10² CFU/ml |
| 13 | Raw milk control |
| 14-19 (Variable 1, Positive | Inoculated raw milk with 10⁴ CFU/ml MAP |
| Control)) | |
| 26-31* (Variable 2) | Inoculated raw milk with 10⁴ CFU/ml MAP |
| | NO LP SYSTEM treatment |
| | 53° C. for 30 min |
| 32-37 (Variable 3) | Inoculated raw milk with 10⁴ CFU/ml MAP |
| | LP SYSTEM 60 treatment |
| | 53° C. for 30 min |
| 38-43(Variable 4) | Inoculated raw milk with 10⁴ CFU/ml MAP |
| | NO LP SYSTEM treatment |
| | 56.5° C. for 30 min |
| 44-49(Variable 5) | Inoculated raw milk with 10⁴ CFU/ml MAP |
| | LP SYSTEM 60 treatment |
| | 56.5° C. for 30 min |
| 50-55 (Variable 6) | Inoculated raw milk with 10⁴ CFU/ml MAP |
| | NO LP SYSTEM treatment |
| | 58.9° C. for 30 min |
| 55-61 (Variable 7) | Inoculated raw milk with 10⁴ CFU/ml MAP |
| | LP SYSTEM 60 treatment |
| | 58.9° C. for 30 min |

*reaction vessels 20-25 were used for an irrelevant enzyme treatment

TABLE 15

| | | Plaque counts/ | | Log₁₀PFU/ | | Estimated CFU | |
|---|---|---|---|---|---|---|---|
| | | 50 ml | | 50 ml | Log | counts/50 ml* | |
| Variables | Description | Rep 1 | Rep 2 | (average) | reduction | Rep 1 | Rep 2 |

| | | | | | | | |
|---|---|---|---|---|---|---|---|
| 1 | Inoculated raw milk | 22000 | 36000 | 4.46 | | 24000 | 39000 |
| (Positive | with 10⁴ CFU · ml | | | | | | |
| Control) | MAP | | | | | | |
| 2 | Inoculated raw milk | 6000 | 13000 | 3.98 | −0.48 | 6500 | 14000 |
| | with 10⁴ CFU · ml | | | | | | |
| | MAP | | | | | | |
| | 53° C. for 30 min | | | | | | |
| 3 | Inoculated raw | 2900 | 3600 | 3.51 | −0.95 | 3200 | 3900 |
| | milk with 10⁴ CFU/ml | | | | | | |
| | MAP | | | | | | |
| | 53° C. for 30 min | | | | | | |
| | LP SYSTEM 60 | | | | | | |
| | treatment | | | | | | |
| 4 | Inoculated raw | 1500 | 3400 | 3.39 | −1.07 | 1600 | 3700 |
| | milk with 10⁴ CFU/ml | | | | | | |
| | MAP | | | | | | |
| | 56.5° C. for 30 min | | | | | | |
| 5 | Inoculated raw | 13 | 17 | 1.18 | −3.28 | 14 | 19 |
| | milk with 10⁴ CFU/ml | | | | | | |
| | MAP | | | | | | |
| | 56.5° for 30 min | | | | | | |
| | LP SYSTEM 60 | | | | | | |
| | treatment | | | | | | |
| 6 | Inoculated raw | 8 | 12 | 1 | −3.36 | 9 | 13 |
| | milk with 10⁴ CFU/ml | | | | | | |
| | MAP | | | | | | |
| | 58.9° C. for 30 min | | | | | | |
| 7 | Inoculated raw | 0 | 0 | 0 | −4.46 | 0 | 0 |
| | milk with 10⁴ CFU/ml | | | | | | |
| | MAP | | | | | | |
| | 58.9° C. for 30 min | | | | | | |
| | LP SYSTEM 60 | | | | | | |
| | treatment | | | | | | |

^{a}Based on FASTPlaque-MAP assay, CFU counts can be estimated using plaques numbers. CFU/ml = No. Plaques * 1.09
^{b}LP SYSTEM 60 = 60% of level used in previous Example 5
Notes:
Article “Rapid assessment of the viability of Mycobacterium avium subsp. paratuberculosis cells after heat treatment, using an optimized phage amplification assay.” Author Irene Grant indicated plaque counts obtained using FASTPlaque-MAP assay were not significantly different from colony counts on HEYM plates.

This study used 60% of the initial LP System level which was used in Example 5 and the results are shown above in Table 15. FASTPlaque-MAP assay showed that a raw milk treatment with the LP System at 56.5° C. for 30 min resulted in a 3.28 log₁₀ CFU/50 ml reduction of MAP over the untreated raw milk control sample. Raw milk with 58.9° C. for 30 min could produce similar reduction for MAP, but LP System 60% eliminated any detectable levels of MAP at 58.9° C. for 30 min.

Although the present invention has been described with reference to preferred embodiments, workers skilled in the art will recognize that changes may be made in form and detail without departing from the spirit and scope of the invention.

What is claimed is:

1. A milk product comprising a milk composition and LP system components, wherein the LP system components comprise lactoperoxidase, glucose oxidase, glucose and an oxidizable agent.

2. The milk product of claim 1 wherein the oxidizable agent is a halide.

3. The milk product of claim 2 wherein the halide is iodide.

4. The milk product of claim 1 wherein the oxidizable agent is thiocyanate.

5. The milk product of claim 1 wherein the milk composition is waste-milk.

6. The milk product of claim 1 wherein the milk composition is colostrum.

7. The milk product of claim 1 wherein the lactoperoxidase is naturally occurring in the milk.

8. The milk product of claim 1 wherein the LP system components further comprise exogenous peroxidases.

9. The milk product of claim 8 wherein the exogenous peroxidases comprise additional lactoperoxidase, horseradish peroxidase, fungal peroxidase or combinations thereof.

10. The milk product of claim 1 wherein the lactoperoxidase system components further comprise hydrogen peroxide, percarbonate, magnesium peroxide, other sources of hydrogen peroxide or combinations thereof.

11. The milk product of claim 1 wherein the amount of glucose added to the milk composition is between about 0.5 g per liter to about 10.0 grams per liter.

12. The milk product of claim 1 wherein the amount of glucose oxidase added to the milk composition is between about 0.01 grams per liter and about 0.1 grams per liter of a 10,000 GOD Units/gram glucose oxidase.

13. The milk product of claim 1 wherein the concentration of the oxidizable agent in the milk product is between about 0.1 ppm and about 10 ppm.

14. The milk product of claim 1 further comprising organic acids, their salts and combinations thereof.

15. The milk product of claim 1 wherein the milk product is pasteurized and the shelf-life of the milk product is greater than about 12 hours at 40° C.

16. The milk product of claim 1 wherein the milk product is pasteurized and the shelf-life of the milk product is greater than about 7 days at 4.5° C.

17. The milk product of claim 1 wherein the milk composition further comprises a milk-balancer.

18. The milk product of claim 17 further comprising nutritional supplements.

19. An LP system activation add pack for milk compositions, the add pack comprising glucose oxidase, glucose and an oxidizable agent wherein addition of the components of the add pack inactivate the bacterial pathogens in the milk composition by activating the LP system.

20. The LP system of claim 19 further comprising lactoperoxidase, horseradish peroxidase, fungal peroxidase, other peroxidases or combinations thereof.

21. The LP system of claim 19 wherein the oxidizable agent is a halide.

22. The LP system of claim 21 wherein the oxidizable agent is iodide.

23. The LP system of claim 19 further comprising hydrogen peroxide, percarbonate, magnesium peroxide or combinations thereof.

24. A method of treating a milk composition comprising activating an enhanced lactoperoxidase system by adding lactoperoxidase system components comprising glucose oxidase, glucose and an oxidizable agent.

25. The method of claim 24 wherein the milk composition is waste-milk.

26. The method of claim 24 wherein the milk composition is colostrum.

27. The method of claim 24 wherein the oxidizable agent is a halide.

28. The method of claim 27 wherein the halide is iodide.

29. The method of claim 24 wherein the oxidizable agent is thiocyanate.

30. The method of claim 24 wherein lactoperoxidase is naturally occurring in the milk.

31. The method of claim 24 further comprising adding exogenous peroxidases in addition to the naturally occurring lactoperoxidase in the milk composition.

32. The method of claim 31 the peroxidases are selected from the group of additional lactoperoxidase, horseradish peroxidase, fungal peroxidase or combinations thereof.

33. The method of claim 24 further comprising addition of hydrogen peroxide, percarbonate, magnesium peroxide, other sources of hydrogen peroxide or combinations thereof.

34. The method of claim 24 wherein the amount of glucose added to the milk composition is between about 0.5 g per liter to about 10.0 grams per liter.

35. The method of claim 24 wherein the amount of glucose oxidase added to treat the milk composition is between about 0.01 grams per liter and about 0.1 grams per liter of a 10,000 GOD Units/gram glucose oxidase.

36. The method of claim 24 wherein the concentration of the oxidizable agent added is between about 0.1 ppm and about 10 ppm.

37. The method of claim 24 further comprising organic acids, their salts and combinations thereof.

38. The method of claim 24 wherein the milk composition is pasteurized and the shelf-life of the milk product is greater than about 12 hours at 40° C.

39. The method of claim 24 wherein the milk composition is pasteurized and the shelf-life of the milk product is greater than about 7 days at 4.5° C.

40. The method of claim 24 wherein activation of the lactoperoxidase system inactivates pathogens.

41. The method of claim 40 wherein the pathogens are E. coli, Salmonella, Clostridium perfringens, Mycobacterium avium subsp. Paratuberculosis (MAP), Mycoplasma bovis and combinations thereof.

42. The method of claim 24 wherein the activation of the lactoperoxidase system reduces the number of pathogens at least about 2-fold.

43. The method of claim 24 wherein the milk composition further comprises a milk-balancer product.

44. The method of claim 43 the milk balancer product comprises nutritional supplements.

45. A method of feeding calves comprising providing a milk composition treated with an enhanced lactoperoxidase system, wherein the treatment comprises activation of an enhanced lactoperoxidase system by addition of enhanced lactoperoxidase system components comprising glucose oxidase, glucose and an oxidizable agent.

46. The method of claim 45 wherein the milk composition is waste-milk.

47. The method of claim 45 wherein the milk composition is colostrum.

48. The method of claim 45 wherein the oxidizable agent is a halide.

49. The method of claim 45 wherein the halide is iodide.

50. The method of claim 45 wherein lactoperoxidase is naturally occurring in the milk composition.

51. The method of claim 45 further comprising adding exogenous peroxidases in addition to the naturally occurring lactoperoxidase in the milk composition.

52. The method of claim 51 wherein the peroxidases are selected from the group of additional lactoperoxidase, horseradish peroxidase, fungal peroxidase or combinations thereof.

53. The method of claim 45 further comprising addition of hydrogen peroxide, percarbonate, magnesium peroxide, other sources of hydrogen peroxide or combinations thereof.

54. The method of claim 45 wherein the milk composition further comprises a milk balancer product.

55. The method of claim 54 wherein the milk balancer product comprises nutritional supplements.

56. A method of reducing the spread of Johne's disease in animals comprising feeding the animals milk products treated with an enhanced lactoperoxidase system wherein the treatment comprises addition of components needed to activate the lactoperoxidase system, the components comprising glucose, glucose oxidase and a halide.

57. The method of claim 56 wherein the milk product is waste-milk.

58. The method of claim 56 wherein the oxidizable agent is a halide.

59. The method of claim 58 wherein the halide is iodide.

60. The method of claim 56 wherein the oxidizable agent is thiocyanate.

61. The method of claim 56 wherein the lactoperoxidase is naturally occurring in the milk.

62. The method of claim 56 further comprising adding exogenous peroxidases in addition to the naturally occurring lactoperoxidase in the milk composition.

63. The method of claim 62 the peroxidases are selected from the group of additional lactoperoxidase, horseradish peroxidase, fungal peroxidase or combinations thereof.

64. The method of claim 56 further comprising addition of hydrogen peroxide, percarbonate, magnesium peroxide, other sources of hydrogen peroxide or combinations thereof.

65. The method of claim 56 wherein the amount of glucose added to the milk composition is between about 0.5 g per liter to about 10.0 grams per liter.

66. The method of claim 56 wherein the amount of glucose oxidase added to treat the milk composition is between about 0.01 grams per liter and about 0.1 grams per liter of a 10,000 GOD Units/gram glucose oxidase.

67. The method of claim 56 wherein the concentration of the oxidizable agent added is between about 0.1 ppm and about 10 ppm.

68. The method of claim 56 further comprising organic acids, their salts or combinations thereof.

69. The method of claim 56 wherein the lactoperoxidase system inactivates Mycobacterium avium subsp. Paratuberculosis (MAP).
